(19) United States
(12) Patent Application Publication
(10) Pub. No.: US 2023/0093191 A1
(43) Pub. Date: Mar. 23, 2023

(54) Biological Material And Preparation Method Therefor

(71) Applicant: VENUS MEDTECH (HANGZHOU) INC., Hangzhou (CN)

(72) Inventors: Yunbing WANG, Hangzhou (CN); Fan YANG, Hangzhou (CN); Gaoyang GUO, Hangzhou (CN)

(73) Assignee: VENUS MEDTECH (HANGZHOU) INC., Hangzhou (CN)

(21) Appl. No.: 18/049,237

(22) Filed: Oct. 24, 2022

Related U.S. Application Data
(63) Continuation of application No. PCT/CN2021/089448, filed on Apr. 23, 2021.

(30) Foreign Application Priority Data
Apr. 24, 2020 (CN) 202010331255.1

(51) Int. Cl. A61L 33/00; C12N 5/071; A61L 27/36; A61L 27/16; A61L 27/54

(52) U.S. Cl. CPC A61L 33/0088; C12N 5/069; A61L 27/3625; A61L 27/16; A61L 27/367; A61L 27/3687; A61L 27/54; A61L 33/0011; C12N 2533/54; C12N 2533/90; A61L 2300/42; A61L 2300/422

(57) ABSTRACT

Provided are an anticoagulation and anticalcification biological material and a preparation method therefor. The preparation method includes the following steps: introducing, on a biological tissue, a polymerizable reactive group, and undergoing free radical copolymerization with a zwitterion. In the present disclosure, by introducing a reactive group capable of free radical polymerization to a biological tissue and undergoing free radical copolymerization with a zwitterionic monomer, collagen in the biological tissue is crosslinked at multiple sites by means of a polymer, thereby achieving sufficient crosslinking within and between collagen fibers, improving the stability of the biological tissue, and prolonging the service life of the biological tissue. Moreover, a zwitterion is introduced to the surface of the biological tissue, to improve the anticoagulation performance, promote the in-situ endothelialization of a biological valve, and prevent the calcium element deposition.

### CROSS REFERENCE OF RELATED APPLICATIONS

The present disclosure is a Continuation application of PCT Application No. PCT/CN2021/089448, filed on Apr. 23, 2021, which claims the priority of Chinese Patent Application No. 202010331255.1, filed on Apr. 24, 2020, the entire contents of which are hereby incorporated by reference.

### TECHNICAL FIELD

The present disclosure relates to the technical field of medical materials, and particularly to an anticoagulation and anticalcification biological material and a preparation method therefor.

### BACKGROUND

Xenogeneic biological tissues have physicochemical properties similar to those of human tissues, and thus are widely used as natural biological materials for implantation in the human body to replace or repair damaged human tissues or organs, particularly used for repairing soft tissues, for example, as artificial blood vessels, artificial valves, vascular patches, dural patches, hernia patches, anti-adhesion membranes, soft tissue fillers, artificial skin and ventricular assist devices. The xenogeneic biological tissues, if directly implanted into the human body without treatment, will be degraded rapidly due to immune rejection, and lose their mechanical performances. For the sake of long-acting performances, the biological tissues are typically chemically cross-linked to remain stable in the human body for a long time. At present, the xenogeneic biological tissues are generally immobilized by cross-linking with glutaraldehyde. However, the biological tissues cross-linked with glutaraldehyde have obvious inflammatory reactions and cross-linking instability, and suffer from the problems of degradation and calcification upon long-term implantation in the body, causing hardening and embrittling, decreased mechanical performances, loss of normal physiological functions of the implanted tissues. Edwards' technique and Medtronic's technique can reduce the calcification of glutaraldehyde-crosslinked biological tissues, but the problems of poor biocompatibility caused by cross-linking with glutaraldehyde cannot be completely eliminated. When the biological tissues are cross-linked with other compounds such as epoxy compounds, carbodiimides, Genipin and other new cross-linking agents, although the biocompatibility and calcification problems can be alleviated, the thrombogenicity problem cannot be overcome. When these tissues are used as a blood contact material, the large number of collagen molecules present in the biological tissue promote the coagulation. Therefore, neither cross-linking with glutaraldehyde nor cross-linking with non-glutaraldehyde can solve the thrombogenesis problem when the biological tissues are used as a blood contact material.

To solve the problems of thrombogenesis and immune rejection associated with the foreign implants in the prior art, the implant surface is usually covalently modified with a functional molecule, to reduce the interaction with the human body, and avoid the coagulation and the attack by the immune system. The covalent modification is beneficial to the maintenance of functional stability for a long period of time, but readily lead to the inactivation of functional molecules. Moreover, heavily depending on the modification site and modification density, the covalent modification often causes the modified surface to fail to achieve a desired anticoagulation or immune evasion effects. The biological tissues are highly sensitive to the modification conditions, and the traditional modification methods often require harsh conditions and have a low modification efficiency. Therefore, the problems of calcification, coagulation, immune reactions and stability associated with the biological tissue implants cannot be solved simultaneously in the prior art; or complex steps are involved when these problems are solved, so biological tissues with excellent comprehensive performances cannot be obtained.

### SUMMARY

In view of the above problems in the prior art, the present disclosure provides a biological material and a preparation method therefor. the biological material has excellent comprehensive performances, exhibiting excellent anticoagulation and anti-calcification performances, immune reactions and stability.

To achieve the above object, the following technical solutions are adopted in the present disclosure.

A method for preparing a biological material includes the following steps:

introducing, on a biological tissue, a polymerizable reactive group, and undergoing free radical copolymerization with a zwitterion. The zwitterionic monomer has a structural formula as below:

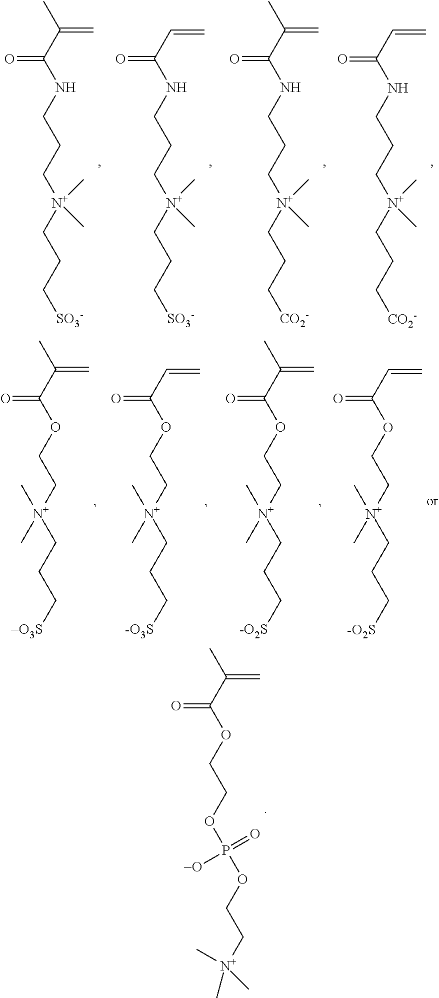

The compounds represented by the above structural formulas are respectively:
N,N-dimethyl-N-methacrylamidopropyl-methanesulfonate;
N,N-dimethyl-N-acrylamidopropyl-methanesulfonate;
N,N-dimethyl-N-methacrylamidopropyl-methanecarboxylate;
N,N-dimethyl-N-methacrylamidopropyl-methanecarboxylate;
[2-(methacryloyloxy)ethyl]dimethyl-(3-sulfopropyl)ammonium hydroxide (SBMA);
[2-(acryloyloxy)ethyl]dimethyl-(3-sulfopropyl)ammonium hydroxide;
3-[[2-(methacryloyloxy)ethyl]dimethyl ammonium]propionate (CBMA);
3-[[2-(acryloyloxy)ethyl]dimethyl ammonium]propionate; and
2-methacryloyloxyethylphosphorylcholine (MPC).

Preferably, the zwitterion is [2-(methylacryloyloxy)ethyl]dimethyl-(3-sulfopropyl)ammonium hydroxide (SBMA), 3-[[2-(Methylacryloyl oxy)ethyl]dimethyl ammonium]propionate (CBMA) or 2-methylacryloyloxyethylphosphorylcholine (MPC).

In some embodiments, the biological tissue introduced with the reactive group is added to a zwitterion solution having a final concentration of 20-500 mM, and an initiator is added to initiate the polymerization, to obtain the anticoagulation and anticalcification biological material.

In some embodiments, the biological tissue introduced with the reactive group is added to a zwitterion solution and soaked at 35 to 40° C., and then an initiator is added to initiate the polymerization, to obtain the anticoagulation and anticalcification biological material. The time of soaking at 35 to 40° C. may be 8 to 24 hrs, for example, 10 to 16 hrs, such as 12 hrs. The specific operation may be soaking overnight.

In some embodiments, the biological tissue is soaked in deionized water, and then the reactive group is added, to provide a concentration of the reactive group of 3-10% by volume.

In some embodiments, the biological tissue is reacted with the reactive group at room temperature for 12 to 36 hrs, and then the biological tissue is washed.

In some embodiments, the biological tissue is soaked in deionized water, and then the reactive group is added to provide a concentration of the reactive group of 4% by volume. and then reacted at room temperature for 24 hrs.

In some embodiments, a zwitterion solution is added to the washed biological tissue, to provide a final concentration of the zwitterion of 500 mM, and the biological tissue is soaked at 37° C. The time of soaking at 37° C. may be 8 to 24 hrs, for example, 10 to 16 hrs, such as 12 hrs. The specific operation may be soaking overnight.

The preparation process is specifically: soaking the biological tissue in deionized water, then adding the reactive group, to provide a concentration of the reactive group of 3 to 10% by volume, reacting at room temperature for 12 to 36 hrs, washing the biological tissue, adding a zwitterion monomer solution to the washed biological tissue to provide a final concentration of the zwitterion monomer of 20 to 500 mM, soaking overnight at 35 to 40° C., and then adding an initiator to initiate the polymerization to obtain the biological material.

The collagen molecule of biological tissues is composed of amino acids, and contains a large amount of free amino groups. The reactive group contains a group that can react with the amino group and also a double bond. The reactive group reacts with free amino group in the biological tissue, to connect the reactive group to the biological tissue through a chemical bond, and the double bond can participate in the subsequent polymerization reaction.

The zwitterion contains a cationic group and an anionic group, and is generally neutrally charged. The zwitterion contains a hydrophilic group such as a sulfonic acid group and a carboxylic acid group, to improve the hydrophilicity. The zwitterion also contains a double bond.

The double bond in the reactive group reacts with the double bond in the zwitterion by free radical reaction in the presence of the initiator, so the reactive group is directly chemically attached to the zwitterion. Moreover, in the presence of the initiator. zwitterion itself will also undergo polymerization, and the collagen molecule in the biological tissue is cross-linked through the polymerization of the zwitterion. Since the polymer with a longer molecular chain structure is cross-linked, long-distance cross-linking is achieved.

Generally, the covalent attachment of a functional molecule to the biological tissue requires the use of a functional group on the functional molecule. The functional group will affect the activity of the functional molecule to some extent. In the present disclosure, the reactive group is grafted to the biological tissue through a chemical bond, and the reactive group is chemically attached to the zwitterion compound through free radical polymerization of the double bond. Free radical polymerization is characterized in that one double bond can be used to react with the zwitterion compound constantly, to achieve an effect that multiple zwitterion compound molecules are introduced by one double bond, so the reactive group is unlikely to be inactivated. Moreover, the free radical polymerization is highly reactive, and can accomplish the covalent bonding of the functional molecule more efficiently compared with general chemical reactions. Further, the reactive group is then added to provide a concentration of the reactive group of 4% by volume. and reacted at room temperature for 24 hrs.

In some embodiments, a zwitterion solution is added to the washed biological tissue, to provide a final concentration of the zwitterion of 500 mM, and the biological tissue is soaked overnight at 37° C.

In some embodiments, the biological tissue is pericardium, aortic root, aortic valve, pulmonary artery root, pulmonary artery valve, tendon, ligament, skin, dura mater, peritoneum, blood vessels, pleura, septum, mitral or tricuspid valve.

In some embodiments, the reactive group is methacrylic anhydride, acrylamide, methacrylamide, acrylate, methacrylate or allyl.

In some embodiments, the zwitterion is attached to the biological tissue through an amide bond at more than two sites of attachment with the biological tissue.

In some embodiments, the zwitterion accounts for 10 to 30% by weight based on the total weight of the anticoagulation and anticalcification biological material; and preferably the zwitterion accounts for 24.6% by weight based on the total weight of the anticoagulation and anticalcification biological material.

In some embodiments, when the initiator initiates the polymerization, the reaction temperature is not higher than 50° C., and preferably 37° C.

In some embodiments, the initiator is a thermal initiator or a photoinitiator. The thermal initiator is an organic peroxide initiator, an inorganic peroxide initiator, an azo initiator, or a redox initiator, and preferably is a redox initiator such as an ammonium persulfate/sodium bisulfite system. The photoinitiator is IrgacureD-2959.

The present application also provides an anticoagulation and anticalcification biological material prepared by any of the above-mentioned preparation methods.

The anticoagulation and anticalcification biological material and the preparation method therefor provided in the present disclosure have the following beneficial effects.

The untreated biological tissues will be degraded under the action of enzymes after implantation, and thus are unable to maintain their functions for a long time. In the present disclosure, a reactive group capable of free radical polymerization is introduced to a biological tissue, which undergoes free radical copolymerization with a zwitterionic monomer, so that collagen in the biological tissue is crosslinked at multiple sites by means of a polymer, thereby achieving sufficient crosslinking within and between collagen fibers, improving the stability of the biological tissue, and prolonging the service life of the biological tissue. Moreover, the zwitterion is introduced to the surface of the biological tissue. The zwitterion is a hydrophilic polymer having anionic and cationic groups, and forms a firm hydration layer on the surface of the biological tissue through strong ionic solvation and hydrogen bonding. The water molecules in the hydration layer are in a highly free state and can hinder the adsorption of proteins, to block the initiation of the downstream coagulation pathway, improve the anticoagulation performance, and promote the in-situ endothelialization of biological valves, thereby preventing the calcium deposition.

### BRIEF DESCRIPTION OF THE DRAWINGS

FIG. 1 is a flow chart illustrating a preparation of a biological tissue.

FIG. 2 is a schematic diagram showing the mechanism of preparation of a functionalized biological tissue.

FIG. 3 is an SEM image showing the platelet adsorption on MPC-20 biological tissue.

FIG. 4 is an SEM image showing the platelet adsorption on MPC-500 biological tissue.

FIG. 5 is an SEM image showing the platelet adsorption on SBMA-20 biological tissue.

FIG. 6 is an SEM image showing the platelet adsorption on SBMA-500 biological tissue.

FIG. 7 is an SEM image showing the platelet adsorption on an unmodified biological tissue.

FIG. 8 is an SEM image showing the platelet adsorption on a glutaraldehyde-crosslinked biological tissue.

FIG. 9 is a fluorescent micrograph showing endothelial cells grown on SBMA-500 biological tissue.

FIG. 10 is a fluorescent micrograph showing endothelial cells grown on an unmodified biological tissue.

FIG. 11 is a fluorescent micrograph showing endothelial cells grown on a glutaraldehyde-crosslinked biological tissue.

### DETAILED DESCRIPTION

### Example 1

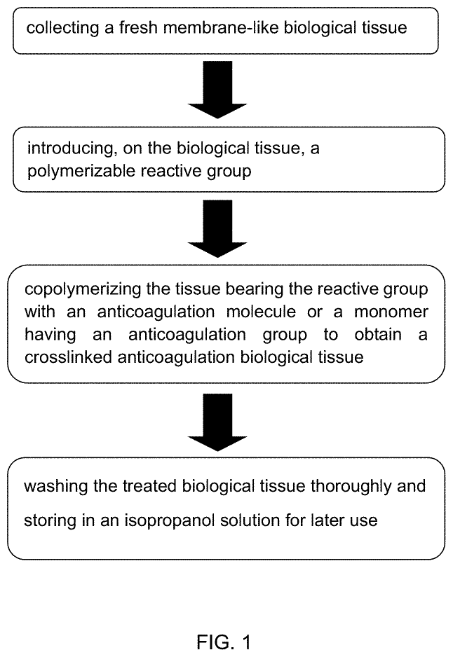

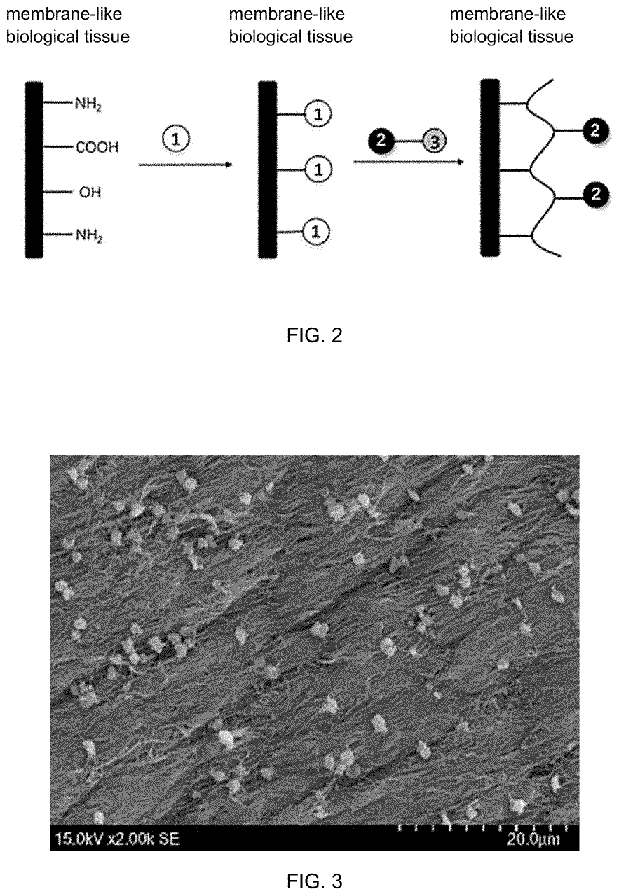

A method for preparing an anticoagulation and anticalcification biological material is provided. The preparation process is shown in FIG. 1, and the mechanism of preparation is shown in FIG. 2, which specifically includes the steps as follows:

washing a fresh porcine pericardium thoroughly with deionized water, and then adding deionized water to completely submerge the tissue; adding methacrylic anhydride dropwise with stirring in an ice bath at 4° C., to obtain a final concentration of the acid anhydride of 4% (v/v), with the solution maintained at pH 7 with a sodium hydroxide solution; and after that, the reaction was continued at room temperature for 24 hrs; thoroughly washing the porcine pericardium modified with methacrylic anhydride, adding the porcine pericardium modified with methacrylic anhydride to a zwitterion monomer solution having a final concentration as shown in a table below, and soaking overnight at 37° C.; then adding an initiator for crosslinking reaction at 37° C. for 24 hrs; obtaining the prepared porcine pericardium. The prepared porcine pericardium was thoroughly washed with deionized water and stored in a 25% isopropanol solution for later use.

Unmodified biological tissue: the unmodified biological tissue was prepared by the steps described above before the initiator is added for crosslinking, and the porcine pericardium is directly cross-linked by adding an initiator without being soaked in a monomer solution. The unmodified porcine pericardium was prepared, and the prepared porcine pericardium was thoroughly washed with deionized water and stored in a 25% isopropanol solution for later use.

Specific formulas are shown below:

| | | | Functional |
|---|---|---|---|
| | Type and | | molecule |
| Sample | concentration | | (MPC/SBMA) |
| name | of monomer | Initiator concentration | content (w/w) |
| MPC-20 | MPC: 20 mM | 50 mM ammonium | 1.6% |
| | | persulfate and 50 mM | |
| | | sodium hydrogen sulfite | |
| MPC-500 | MPC: 500 mM | 50 mM ammonium | 15.6% |
| | | persulfate and 50 mM | |
| | | sodium hydrogen sulfite | |
| SBMA-20 | SBMA: 20 mM | 50 mM ammonium | 5.2% |
| | | persulfate and 50 mM | |
| | | sodium hydrogen sulfite | |
| SBMA-500 | SBMA: 500 mM | 50 mM ammonium | 24.6% |
| | | persulfate and 50 mM | |
| | | sodium hydrogen sulfite | |
| Unmodified | 0 | 50 mM ammonium | 0%* |
| | | persulfate and 50 mM | |
| | | sodium hydrogen sulfite | |

Note:
SBMA: [2-(methylacryloyloxy)ethyl]dimethyl-(3-sulfopropyl)ammonium hydroxide;
MPC: 2-methylacryloyloxyethyl phosphorylcholine;
*the unmodified is used as a reference line, set as 0%; and the initiator concentration is the concentration initially added to the reaction system.

The above-mentioned functional molecule content is derived from analyzing by ICP-OES the content of sulfur or phosphorus in the biological material after freeze-dried since phosphorus element are contained in the functional molecule MPC and sulfur element are contained in SBMA, both of which are hardly found in the biological material. Thus, the degree of modification with the functional molecule on the biological tissue can be obtained, which facilitates the screening of experimental conditions.

The specific conversion formula is as follows:

$$Functional molecule content = \frac{{C}_{S ( P )} \times 10 / {M}_{S ( P )} \times {M}_{SBMA ( MPC )}}{W} \times 100 \%$$

wherein C_{S(P)} (mg/L) is the concentration of sulfur or phosphorus in solution measured by ICP-OES, M_{S(P)} is the relative atomic mass of sulfur or phosphorus, M_{SBMA(MPC)} is the relative molecular mass of the functional molecule, and W (mg) is the dry weight of the biological material.

As can be seen from the above table, compared with the control group, the biological material prepared by the present disclosure shows that the biological tissue is well modified with the functional molecule, particularly, the SBMA-500 group has the highest degree of modification.

### Test Example 1: Stability of Biological Tissue

The obtained biological material was cut into small pieces of 3-8 mg, aspirated with filter paper to remove water on the surface, and then placed in an aluminum crucible and sealed. The crucible with the sample was placed in the instrument and the thermal denaturation temperature was analyzed by differential scanning calorimetry, to characterize the stability of the biological tissue. The results are shown below:

| | Thermal denaturation |
|---|---|
| Sample name | temperature (° C.) |
| SBMA-500 | 80.15 |
| Fresh tissue | 68.88 |

As can be seen from the above table, the biological material prepared by the present disclosure has a higher temperature resistance compared with fresh tissue, indicating that the biological tissue has excellent stability.

### Test Example 2: Anticoagulation Performance of Biological Tissue

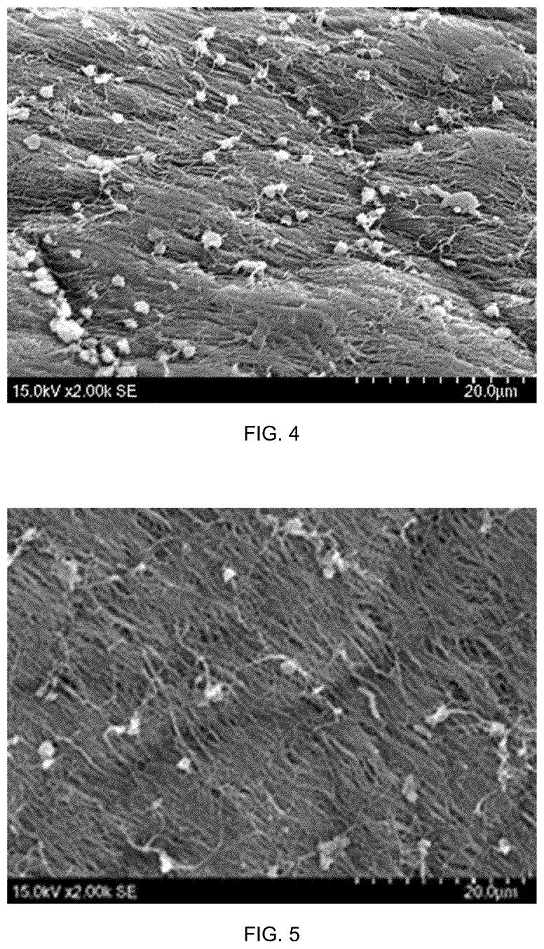

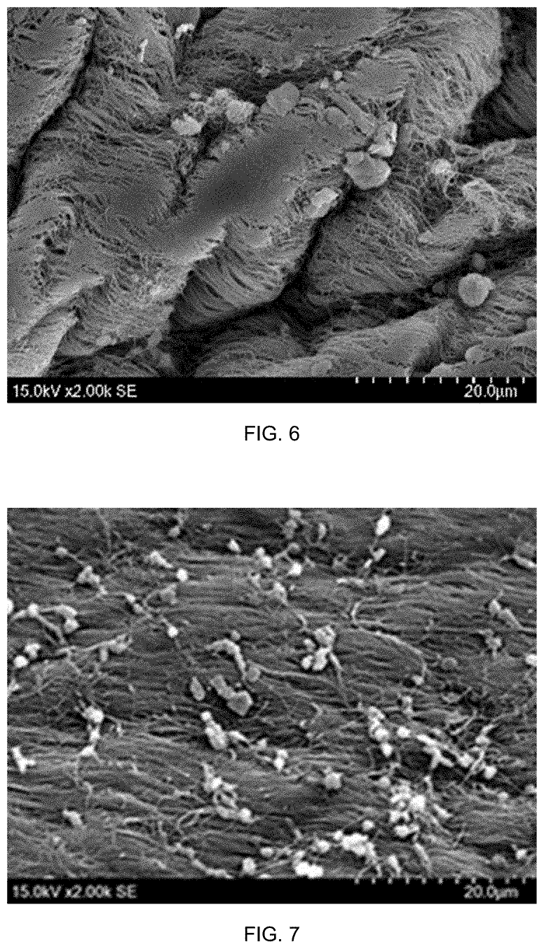

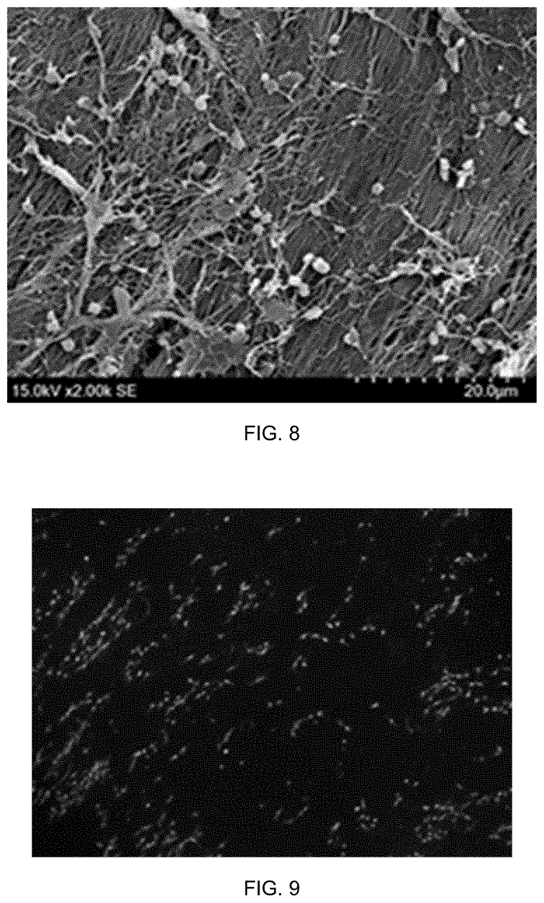

The prepared biological material was washed, cut to have an appropriate size, and incubated with platelet-rich plasma for one hr. The amount of lactate dehydrogenase (LDH) after lysis of platelets adhering to the material was measured to express the amount of platelet adhered. The results are shown in a table below. The adhesion of platelets on the material was observed under SEM. The results are specifically shown in FIGS. 3-8.

| | Absorbency value |
|---|---|
| Sample name | (490 nm) |
| MPC-20 | 0.30 |
| MPC-500 | 0.29 |
| SBMA-20 | 0.24 |
| SBMA-500 | 0.110 |
| Unmodified | 0.32 |
| Crosslinked with | 0.35 |
| glutaraldehyde | |

As can be seen from the above table, a higher absorbency value at 490 nm indicates a higher lactate dehydrogenase content, that is, the amount of platelets adhered to the material is higher. Therefore, as can be seen from the above table, the anticoagulation effect of the biological material prepared by the present disclosure is apparently better than those of unmodified biological tissue and glutaraldehyde-crosslinked biological tissue (prepared by immersing porcine pericardium in 0.625 vol % of glutaraldehyde aqueous solution, crosslinking for 24 hrs at pH 7.4 and room temperature, and then dehydrating and drying). The anticoagulation effect of the biological tissue modified with SBMA is better than that of the biological tissue modified with MPC, and the anticoagulation effect of the biological tissue with a high grafting degree of SBMA is the best.

The qualitative observation results under SEM in FIGS. 3-8 are consistent with the quantitative measurement results of LDH.

### Test Example 3. Characterization of Anti-Calcification Performance and Immune Reaction of Biological Tissue

The prepared biological material was cut into pieces of 1 square centimeter, subcutaneously implanted in SD rats, and removed after 90 days. The calcium content in the samples was measured by ICP-OES. The results are shown below:

| Sample name | Calcium content (g/kg) |
|---|---|
| SBMA-500 | 12.8 |
| Unmodified | 38.4 |
| Crosslinked with | 117.3 |
| glutaraldehyde | |

As can be seen from the above table, the calcification of the biological tissue modified with SBMA-500 was significantly lower than those of unmodified biological tissue and glutaraldehyde-crosslinked biological tissue.

### Test Example 4: Recellularization Performance of Biological Tissue

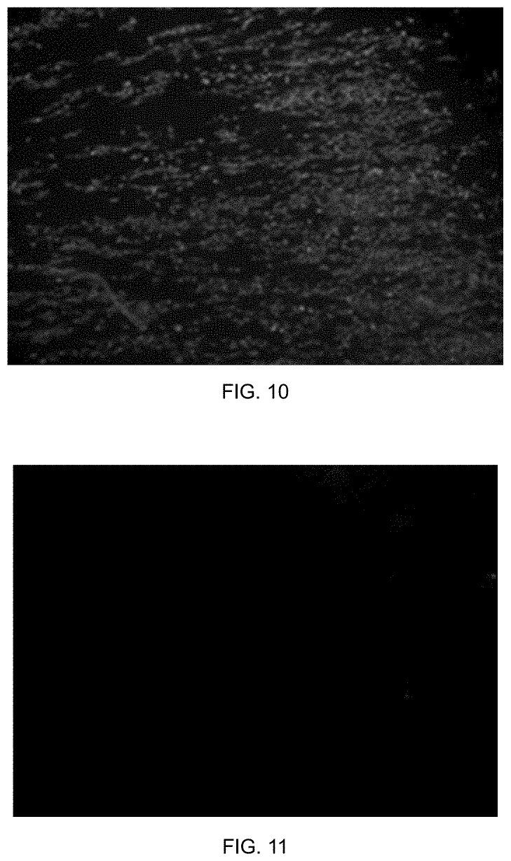

The surface of the prepared biological material was seeded with endothelial cells, incubated for 3 days, and then stained with DAPI and FITC labeled phalloidin. The results are shown in FIGS. 9-11.

As can be seen from FIGS. 9-11, the growth of endothelial cells on the biological tissue modified with SBMA-500 was better than those on unmodified biological tissue and glutaraldehyde-crosslinked biological tissue.

### Test Example 5. Implantation of Artificial Blood Vessels

The prepared biological material was rolled into a tube with a diameter of about 2 mm and the junction was bonded with a medical adhesive, aspirated with filter paper to remove water on the surface, weighed and recorded. A rabbit carotid artery having a length of approximately 2 cm was replaced by the tube using vascular anastomosis. After implantation for a period of time, it was retracted, observed, aspirated with filter paper to remove water on the material, and weighed again. The weight gain was calculated, which is the amount of thrombus produced. The result is shown below.

| | Initial weight | Final | Amount of thrombus |
|---|---|---|---|
| Sample name | (mg) | weight (mg) | produced (mg) |
| SBMA-500 | 80.9 | 83.6 | 2.7 |
| Unmodified | 78.6 | 262.4 | 183.8 |
| Crosslinked with | 82.5 | 295.3 | 212.8 |
| glutaraldehyde | | | |

From the above table, it can be seen that for glutaraldehyde-crosslinked biological tissue and unmodified biological tissue, thrombus of more than 2 times of the tissue weight is produced, but almost no thrombus is produced for SBMA-500.

What is claimed is:

1. A method for preparing a biological material, comprising following steps: introducing, on a biological tissue, a polymerizable reactive group, and undergoing free radical copolymerization with a zwitterion, wherein the zwitterion has a specific structure as below:

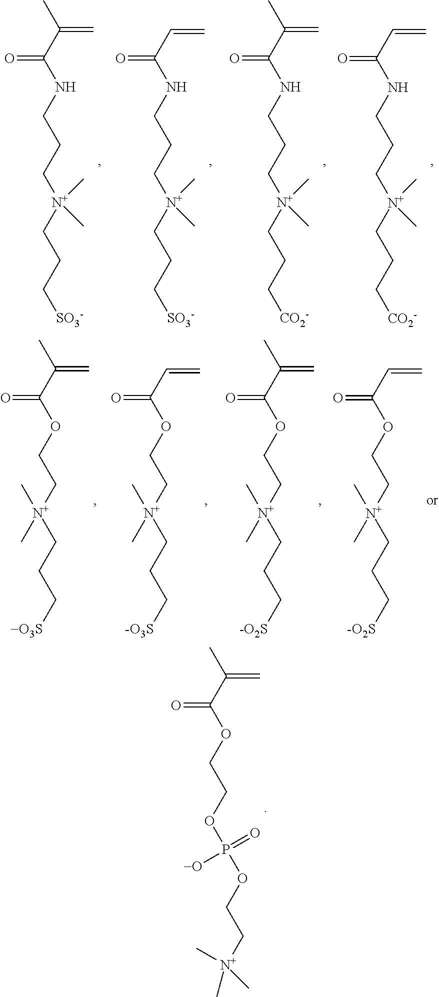

2. The method for preparing a biological material according to claim 1, wherein the biological tissue introduced with the reactive group is added to a zwitterion solution having a final concentration of 20 to 500 mM, and an initiator is added to initiate the polymerization, to obtain the biological material.

3. The method for preparing a biological material according to claim 1, wherein the biological tissue introduced with the reactive group is added to a zwitterion solution and soaked at 35 to 40° C., and an initiator is added to initiate the polymerization, to obtain the biological material.

4. The method for preparing a biological material according to claim 1, wherein the biological tissue is soaked in deionized water, and then the reactive group is added, to provide a concentration of the reactive group of 3-10% by volume.

5. The method for preparing a biological material according to claim 4, wherein the biological tissue is reacted with the reactive group at room temperature for 12 to 36 hrs, and then the biological tissue is washed.

6. The method for preparing a biological material according to claim 4, wherein the biological tissue is soaked in deionized water, and then the reactive group is added to provide a concentration of the reactive group of 4% by volume. and then reacted at room temperature for 24 hrs.

7. The method for preparing a biological material according to claim 1, wherein the zwitterion solution is added to the washed biological tissue, to provide a final concentration of the zwitterion of 500 mM, and the biological tissue is soaked at 37° C.

8. The method for preparing a biological material according to claim 1, wherein the biological tissue is pericardium, aortic root, aortic valve, pulmonary artery root, pulmonary artery valve, tendon, ligament, skin, dura mater, peritoneum, blood vessels, pleura, septum, mitral or tricuspid valve.

9. The method for preparing a biological material according to claim 1, wherein the reactive group is methacrylic anhydride, acrylamide, methacrylamide, acrylate, methacrylate or allyl.

10. The method for preparing a biological material according to claim 1, wherein the zwitterion accounts for 1 to 30% by weight based on a total weight of the biological material.

11. The method for preparing a biological material according to claim 2, wherein when the initiator initiates the polymerization, a reaction temperature is not higher than 50° C.

12. The method for preparing a biological material according to claim 2, wherein the initiator is a thermal initiator or a photoinitiator.

13. A biological material prepared by the preparation method according to claim 1.

14. A biological material prepared by the preparation method according to claim 2.

15. A biological material prepared by the preparation method according to claim 3.

16. A biological material prepared by the preparation method according to claim 4.

17. A biological valve prepared by the preparation method according to claim 1.

18. A biological valve prepared by the preparation method according to claim 2.

19. A biological valve prepared by the preparation method according to claim 3.

20. A biological valve prepared by the preparation method according to claim 4.
